(19) United States
(12) Patent Application Publication
(10) Pub. No.: US 2024/0041966 A1
(43) Pub. Date: Feb. 8, 2024

(54) A NUTRACEUTICALS FORMULATION FOR HYPERTENSION WITH ENHANCED ORGANOLEPTIC PROPERTIES

(71) Applicant: DiaBliss Consumer Products Pvt Ltd, Chennai (IN)

(72) Inventors: Vr. RAMANATHAN, Chennai (IN); Siva VALLABHANENI, Chennai (IN); V. RAJALAKSHMI, Chennai (IN)

(73) Assignee: DiaBliss Consumer Products Pvt Ltd, Chennai, TN (IN)

(21) Appl. No.: 18/258,639

(22) PCT Filed: Dec. 22, 2021
(86) PCT No.: PCT/IN2021/051197

§ 371 Date: Jun. 21, 2023

(30) Foreign Application Priority Data
Dec. 22, 2020 (IN) 202041055870

(51) Int. Cl. A61K 36/54; A61K 36/71; A61K 36/9066; A61K 36/9068; A61K 36/61; A61K 36/55; A61K 36/484; A61K 36/235; A61P 9/12

(52) U.S. Cl. CPC A61K 36/54; A61K 36/71; A61K 36/9066; A61K 36/9068; A61K 36/61; A61K 36/55; A61K 36/484; A61K 36/235; A61P 9/12

(57) ABSTRACT

Nutraceutical formulations, used for prevention, cure, treatment and/or management of hypertension and/or associated condition(s), with enhanced organoleptic properties as monotherapy, combination and/or dual therapy. The formulations have elements of biological origin more particularly a herbal formulation(s).

### FIELD OF THE INVENTION

The subject matter described herein, in general, relates to a nutraceuticals formulation, which could be used for treatment and/or management of hypertension and/or associated condition(s), with an enhanced organoleptic properties. Said formulation have elements of biological origin more particularly a herbal formulation(s).

### BACKGROUND OF THE INVENTION

Blood pressure is the pressure of blood pushing against the walls of the arteries. Arteries carry blood from the heart to other parts of the body. Blood pressure normally rises and falls throughout the day, but it can damage your heart and cause health problems if it stays high for a long time. Hypertension, also called high blood pressure, is blood pressure that is higher than normal.

Blood pressure readings are given in two numbers. The top number is the maximum pressure the heart exerts while beating (systolic pressure). The bottom number is the amount of pressure in the arteries between beats (diastolic pressure).

The top number (systolic) minus the bottom number (diastolic) gives one's pulse pressure. For example, if the resting blood pressure is 120/80 millimeters of mercury (mm Hg), the pulse pressure is 40 mm Hg which is considered a normal and healthy pulse pressure. Generally, a pulse pressure greater than 40 mm Hg is abnormal. Measuring pulse pressure may help predict if someone is at risk for a heart event, including a heart attack or stroke. If pulse pressure is greater than 60 it's considered a risk factor for cardiovascular disease, especially for older adults.

Stiffness of the body's largest artery, the aorta, is the leading cause of increased pulse pressure in older adults. High blood pressure or fatty deposits on the walls of the arteries (atherosclerosis) can make your arteries stiff. The greater your pulse pressure, the stiffer and more damaged the blood vessels are thought to be.

The largest blood vessels are arteries and veins, which have a thick, tough wall of connective tissue and many layers of smooth muscle cells. The wall is lined by an exceedingly thin single sheet of endothelial cells, the endothelium. Endothelial cells form a single cell layer that lines all blood vessels and regulates exchanges between the bloodstream and the surrounding tissues.

Arterial stiffening, at least in part, reflects gradual fragmentation and loss of elastin fibers and accumulation of stiffer collagen fibers in the arterial wall. Increased arterial stiffness is closely linked to increased risk of hypertension and other diseases, such as chronic kidney disease and stroke.

The pathogenesis of atherosclerosis remains incompletely understood, but inflammation is thought to play an important role. Several studies have demonstrated that serum levels of the acute phase protein, C-reactive protein (CRP), independently predict outcome in patients with cardiovascular disease and in apparently healthy individuals. Levels of CRP also correlate with endothelial function, an independent predictor of cardiovascular risk, in patients with coronary artery disease. Moreover, CRP has direct proinflammatory effects on human endothelial cells in vitro and can induce endothelial dysfunction.

In 2017, the American College of Cardiology and the American Heart Association published new guidelines for hypertension management which are summarized in Table 1 below:

TABLE 1

| | | | | Diastolic | |
|---|---|---|---|---|---|
| Blood Pressure | Systolic Blood | | | Blood | |
| Category | Pressure | | | Pressure | |
| Normal | <120 | mm Hg | and | <80 | mm Hg |
| Elevated | 120-129 | mm Hg | and | <80 | mm Hg |

Hypertension

| | | | | | |
|---|---|---|---|---|---|
| Stage 1 | 130-139 | mm Hg | or | 80-89 | mm Hg |
| Stage 2 | ≥140 | mm Hg | or | ≥90 | mm Hg |

Hypertension is the leading cause of cardiovascular disease and premature death worldwide. Owing to the widespread use of antihypertensive medications, global mean blood pressure (BP) has remained constant or has decreased slightly over the past four decades. By contrast, the prevalence of hypertension has increased, especially in low- and middle-income countries (LMICs). Estimates suggest that 31.1% of adults (1.39 billion) worldwide had hypertension in 2010. The prevalence of hypertension among adults was higher in LMICs (31.5%, 1.04 billion people) than in high-income countries (28.5%, 349 million people).

In 2018, nearly half a million deaths in the United States included hypertension as a primary or contributing cause. Nearly half of adults in the United States (108 million, or 45%) have hypertension defined as a systolic blood pressure 130 mm Hg or a diastolic blood pressure 80 mm Hg or are taking medication for hypertension. Only about 1 in 4 adults (24%) with hypertension have their condition under control. About half of adults (45%) with uncontrolled hypertension have a blood pressure of 140/90 mm Hg or higher. This includes 37 million U.S. adults.

About 33% urban and 25% rural Indians are hypertensive. Of these, 25% rural and 42% urban Indians are aware of their hypertensive status. Only 25% rural and 38% of urban Indians are being treated for hypertension. One-tenth of rural and one-fifth of urban Indian hypertensive population have their BP under control. Case-control studies have reported that hypertension is most important risk factor for Cardio Vascular Disease (CVD) in India. These studies estimated that hypertension led to 1.6 million deaths and 33.9 million disability-adjusted life years in 2015 and is most important cause of mortality as well as disease burden in India.

Variations in the levels of risk factors for hypertension, such as high sodium intake, low potassium intake, obesity, alcohol consumption, physical inactivity and unhealthy diet, may explain some of the regional heterogeneity in hypertension prevalence. Despite the increasing prevalence, the proportions of hypertension awareness, treatment and BP control are low, particularly in LMICs, and few comprehensive assessments of the economic impact of hypertension exist.

Many blood pressure medications, known as antihypertensives, are available to lower high blood pressure (HBP or hypertension). There are a variety of classes of high blood pressure medications and they include a number of different drugs: Diuretics, Beta-blockers, ACE inhibitors, Angiotensin II receptor blockers, Calcium channel blockers, Alpha blockers, Alpha-2 Receptor Agonists, Combined alpha and beta-blockers, Central agonists, Peripheral adrenergic inhibitors, Vasodilators and/or their combination.

Diuretics help the body get rid of excess sodium (salt) and water and help control blood pressure. They are often used in combination with additional prescription therapies. Some of these drugs may decrease body's supply of the mineral potassium. Symptoms such as weakness, leg cramps or being tired may result. Eating foods containing potassium may help prevent significant potassium loss.

Beta-blockers reduce the heart rate, the heart's workload and the heart's output of blood, which lowers blood pressure. Some noted possible side effects of beta-blockers include insomnia, Cold hands and feet, Tiredness or depression, Slow heartbeat, Symptoms of asthma and impotence.

Angiotensin is a chemical that causes the arteries to become narrow, especially in the kidneys but also throughout the body. ACE stands for Angiotensin-converting enzyme. ACE inhibitors help the body produce less angiotensin, which helps the blood vessels relax and open up, which, in turn, lowers blood pressure. Some noted possible side effects of ACE inhibitors are Skin rash, Loss of taste, Chronic dry, hacking cough, in rare instances kidney damage. Women who are taking ACE inhibitors or ARBs for high blood pressure should not become pregnant while on this class of drugs. These drugs have been shown to be dangerous to both mother and baby during pregnancy. They can cause low blood pressure, severe kidney failure, excess potassium (hyperkalemia) and even death of the newborn.

Angiotensin II receptor blockers are drugs that block the effects of angiotensin, a chemical that causes the arteries to become narrow. Angiotensin needs a receptor—like a chemical “slot” to fit into or bind with—in order to constrict the blood vessel. ARBs block the receptors so the angiotensin fails to constrict the blood vessel. This means blood vessels stay open and blood pressure is reduced. Some noted possible side effects of Angiotensin II receptor blockers include occasional dizziness. ARBs are not recommended to be used during pregnancy as they act directly on the renin-angiotensin system and can cause injury or even death to a developing fetus.

Calcium channel blockers prevents calcium from entering the smooth muscle cells of the heart and arteries. When calcium enters these cells, it causes a stronger and harder contraction, so by decreasing the calcium, the hearts' contraction is not as forceful. Calcium channel blockers relax and open up narrowed blood vessels, reduce heart rate and lower blood pressure. Some noted possible side effects of calcium channel blockers are Palpitations, Swollen ankles, Constipation, Headache, Dizziness.

Alpha blockers reduce the arteries' resistance, relaxing the muscle tone of the vascular walls. Some noted possible side effects of alpha blockers include fast heart rate, dizziness and a drop in blood pressure when standing up.

Alpha-2 Receptor Agonists are drugs reduce blood pressure by decreasing the activity of the sympathetic (adrenaline-producing) portion of the involuntary nervous system.

Combined alpha and beta-blockers are used as an IV drip for those patients experiencing a hypertensive crisis. A noted possible side effect of combined alpha and beta-blockers is it may cause a drop in blood pressure when one stands up.

Central agonists also help decrease the blood vessels' ability to tense up or contract. The central agonists follow a different nerve pathway than the alpha and beta-blockers, but accomplish the same goal of blood pressure reduction. Some noted possible side effects of central agonists include a greater drop in blood pressure when one is in an upright position (standing or walking), and it may make one feel weak or faint if the pressure has been lowered too far. This drug may also cause drowsiness or sluggishness, dryness of the mouth, fever or anemia. Male patients may experience impotence.

Peripheral adrenergic inhibitors reduce blood pressure by blocking neurotransmitters in the brain. This blocks the smooth muscles from getting the “message” to constrict. These drugs are rarely used unless other medications don't help. Some noted possible side effects of peripheral adrenergic inhibitors include stuffy nose, diarrhea or heartburn. Some drugs in this class of medication may cause some diarrhea, which may persist in some people. These drugs reduce blood pressure more when one stands. Consequently, one may get dizzy and lightheaded and feel weak when you get out of bed in the morning or stand up suddenly.

Blood vessel dilators, or vasodilators, can cause the muscle in the walls of the blood vessels (especially the arterioles) to relax, allowing the vessel to dilate (widen). This allows blood to flow through better. Some noted possible side effects of vasodilators are headaches, swelling around the eyes, heart palpitations or aches and pains in the joints. Usually none of these symptoms are severe, and most will go away after a few weeks of treatment.

As it can be seen there are many classes of drugs and each of these classes of drugs have their own side effects. Most hypertensive patients are administered combination of drugs by physicians in order to optimize the reduction in blood pressure and also to manage the side effects. According to paper titled, “Use of blood pressure lowering drugs in the prevention of cardiovascular disease: meta-analysis of 147 randomised trials in the context of expectations from prospective epidemiological studies” by M. R. Law, J. K. Morris and N. J. Wald, BMJ 2009;338:b1665, the proportional reduction in CHD events and stroke for a given reduction in blood pressure, an approximate halving in risk for each 10 mm Hg diastolic reduction, is the same in people with and without a history of vascular disease and in people without high blood pressure as well as in those with high blood pressure.” Among the various classes of drugs analysed through meta studies, the five main classes of blood pressure lowering drugs (thiazides, (3 blockers, angiotensin converting enzyme inhibitors, angiotensin receptor blockers, and calcium channel blockers) were similarly effective (within a few percentage points) in preventing CHD events and strokes, with the exception that calcium channel blockers had a greater preventive effect on stroke. Typical reductions in Systolic and Diastolic BP levels were 10 mm Hg and 5 mm Hg respectively. Thus, currently no medicine or regimen is available for an effective treatment and/or management or cure of hypertension and associated conditions, the available medicines though manage it to an extent but also has many side effects. Furthermore, prevention, treatment, cure and/or management of hypertension for a diabetes patients is still very complex and crucial. There is a need for treatment, cure and/or effective management of hypertension and/or diabetes with reduced number of medicines, organoleptic formulations for improved life style and thus increased patient compliance.

### SUMMARY OF THE INVENTION

In the last three decades, a lot of concerted efforts have been channeled into researching into local plants with hypotensive and antihypertensive therapeutic values. The hypotensive and antihypertensive effects of some of these medicinal plants have been validated and others disproved.

It is an object of the present invention to provide for a water based herbal formulation that addresses hypertension among human subjects. The product formulation is prepared using the embodiments described in the Indian Patent application no: 202041041780.

A herbal Formulation for the treatment and/or management of hypertension comprising essentially of a therapeutically effective amount of Nigella sativa, Cinnamomum verum, Curcuma longa, Zingiber officinale, Elettaria cardamom, Myristica fragrans, Syzygium aromaticum, Psidium guajava, Linum usitatissimum, Cuminum cyminum, Glycyrrhiza glabra, Trigonella foenum-graecum, Foeniculum vulgare.

As one of the embodiment, the aforesaid herbal formulation broadly comprises of the herbal constituents in the range of Nigella sativa 0-10.0%, Cinnamomum verum 0-5.0%, Curcuma longa 0-7.0%, Zingiber officinale 1.0-7.75%, Elettaria cardamom 0.5-10%, Myristica fragrans 2.0-10.5%, Syzygium aromaticum 1.25-9.5%, Psidium guajava 10.0-65.0%, Linum usitatissimum 8.0-55.0%, Cuminum cyminum 0.5-12.5%, Glycyrrhiza glabra 0.5-1.5%, Trigonella foenum-graecum 0-6.5%, Foeniculum vulgare 0-7.5%.

As one of the embodiment, the herbal formulation of the present invention could be manufactured using parts of the plants used for preparing the extracts are Nigella sativa Seeds, Cinnamomum verum Seeds, Curcuma longa Root, Zingiber officinale Root, Elettaria cardamom Seeds, Myristica fragrans Seed, Syzygium aromaticum Flower Buds, Psidium guajava Leaves, Linum usitatissimum Seeds, Cuminum cyminum Seeds, Glycyrrhiza glabra Root, Trigonella foenum-graecum Seeds, Foeniculum vulgare Seeds.

As another embodiment the herbal formulation of the present invention comprises of the herbal constituents present in the range of Nigella sativa 1.5%, Cinnamomum verum 2.5%, Curcuma longa 1.0%, Zingiber officinale 3.0%, Elettaria cardamom 3.2%, Myristica fragrans 2.8%, Syzygium aromaticum 3.0%, Psidium guajava 43.5%, Linum usitatissimum 31.5%, Cuminum cyminum 4.5%, Glycyrrhiza glabra 1.5%, Trigonella foenum-graecum 1.2%, Foeniculum vulgare 1.3%.

### BRIEF DESCRIPTION OF ACCOMPANYING DRAWINGS

FIG. 1 is plot of the average systolic blood pressure of all subjects, old and newly diagnosed hypertensives during the course of the clinical trial.

FIG. 2 is plot of the average systolic blood pressure of all subjects, old and newly diagnosed hypertensives during the course of the clinical trial.

FIG. 3 is plot of the C-Reactive protein of all subjects at the start of the clinical trial and at the end of the clinical trial (day 180).

FIG. 4 is plot of the Systolic blood pressure values in mm Hg for all the subjects at the start of the clinical trial (Day 0) and at the end of the clinical trial (day 180)

FIG. 5 is plot of the Systolic blood pressure values in mm Hg for all the subjects at the start of the clinical trial (Day 0) and at the end of the clinical trial (day 180)

FIG. 6 is plot of the glycated hemoglobin (HbA1c) of all the clinical trial participants at the start of the clinical trial (baseline) and at the end of the clinical trial (day 180)

### DETAILED DESCRIPTION

The following presents a detailed description of various embodiments of the present subject matter with reference to the accompanying drawings. The embodiments of the present subject matter are described in detail with reference to the accompanying drawings. However, the present subject matter is not limited to these embodiments which are only provided to explain more clearly the present subject matter to a person skilled in the art of the present disclosure.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements, and/or components, but do not preclude the presence or addition of one or more other features, steps, operations, elements, components, and/or groups thereof. As used herein, the term “and/or” includes any and all combinations of one or more of the described ingredients having similar properties.

The subject matter described herein relates to a method of improving functional and organoleptic properties of natural products and nutraceutical composition(s) thereof for the better treatment, cure and/or management of hypertension.

The foregoing and further objects, features and advantages of the present subject matter will become apparent from the following description of exemplary embodiments with reference to the accompanying drawings.

It is to be noted, however, that the appended drawings illustrate only typical embodiments of the present subject matter, and are therefore, not to be considered for limiting of its scope, for the subject matter may admit to other equally effective embodiments.

The present invention relates to a nutraceutical formulation; a herbal Formulation for the prevention, treatment, cure and/or management of hypertension comprising essentially of a therapeutically effective amount of Nigella sativa, Cinnamomum verum, Curcuma longa, Zingiber officinale, Elettaria cardamom, Myristica fragrans, Syzygium aromaticum, Psidium guajava, Linum usitatissimum, Cuminum cyminum, Glycyrrhiza glabra, Trigonella foenum-graecum, Foeniculum vulgare.

As per another embodiment, the aforesaid herbal formulation broadly comprises of the herbal constituents in the range of Nigella sativa 0-10.0%, Cinnamomum verum 0-5.0%, Curcuma longa 0-7.0%, Zingiber officinale 1.0-7.75%, Elettaria cardamom 0.5-10%, Myristica fragrans 2.0-10.5%, Syzygium aromaticum 1.25-9.5%, Psidium guajava 10.0-65.0%, Linum usitatissimum 8.0-55.0%, Cuminum cyminum 0.5-12.5%, Glycyrrhiza glabra 0.5-1.5%, Trigonella foenum-graecum 0-6.5%, Foeniculum vulgare 0-7.5%.

As per another embodiment, the herbal formulation of the present invention could be manufactured using parts of the plants used for preparing the extracts are Nigella sativa Seeds, Cinnamomum verum Seeds, Curcuma longa Root, Zingiber officinale Root, Elettaria cardamom Seeds, Myristica fragrans Seed, Syzygium aromaticum Flower Buds, Psidium guajava Leaves, Linum usitatissimum Seeds, Cuminum cyminum Seeds, Glycyrrhiza glabra Root, Trigonella foenum-graecum Seeds, Foeniculum vulgare Seeds.

As yet another embodiment the herbal formulation of the present invention comprises of the herbal constituents present in the ratio of Nigella sativa 1.5%, Cinnamomum verum 2.5%, Curcuma longa 1.0%, Zingiber officinale 3.0%, Elettaria cardamom 3.2%, Myristica fragrans 2.8%, Syzygium aromaticum 3.0%, Psidium guajava 43.5%, Linum usitatissimum 31.5%, Cuminum cyminum 4.5%, Glycyrrhiza glabra 1.5%, Trigonella foenum-graecum 1.2%, Foeniculum vulgare 1.3%.

As yet another embodiment of the present invention, the present formulation has shown very significant results in prevention, treatment, cure and/or management of hypertension associated conditions including of diabetic patients. High blood pressure (hypertension) can lead to many complications of diabetes, including diabetic eye disease and kidney disease, most people with diabetes will eventually have high blood pressure, along with other heart and circulation problems. Further, Diabetes damages arteries and makes them targets for hardening, called atherosclerosis. That can cause high blood pressure, which if not treated, can lead to trouble including blood vessel damage, heart attack, and kidney failure.

As per scientific publication, “Amanda N. Long, Doand Samuel Dagogo-Jack, MD, The Comorbidities of Diabetes and Hypertension: Mechanisms and Approach to Target Organ Protection, J ClinHypertens (Greenwich). 2011 April; 13(4): 244-251”, these two giant disorders often come hand in hand with each other. Up to 75% of adults with diabetes also have hypertension and patients with hypertension alone often show evidence of insulin resistance. Thus, hypertension and diabetes are common, intertwined conditions that share a significant overlap in underlying risk factors (including ethnicity, familial, dyslipidemia and lifestyle determinants) and complications.

High blood pressure is twice as likely to strike a person with diabetes than a person without diabetes. In fact, a person with diabetes and high blood pressure is four times as likely to develop heart disease than someone who does not have either of the conditions. It is therefore important to control both diabetes and hypertension among individuals.

The herbal Formulation of the present invention is used for the treatment and management of hypertension. It also has shown significant results in associated conditions of hypertension like for the prevention, management or cure of atherosclerosis, maintaining blood level of CRP, maintaining vascular homeostasis, reduction in cardiac oxidative stress, heart endurance and angiotensin-converting enzyme activity.

As yet another embodiment of the present invention, the herbal formulation may be used for monotherapy or as a combination or dual therapy with diabetic, hypertension drugs or other drugs.

As yet another embodiment of the herbal formulation demonstrates significant improvement and maintenance of heart endurance.

### Multi Herbal System Requirement

Most single herbs and spices to provide a particular functionality exhibit certain predominant mechanism of action. If we take management of hypertension in the human body, there are multiple processes that impact blood pressure including arterial stiffness, blood rheology & viscosity, arterial plaque blockages, psychosomatic factors such as stress and depression, metabolic factors, obesity. These are complex processes known to those skilled in human physiology. A single herb or spice or plant may deliver one or more active ingredients that can impact a particular hypertension pathway. Due to diversity of physiology among humans, there could be one or more pathways that are deficient in the human blood pressure regulation pathway. By consuming one herb they may or may not effectively address the needs of a particular individual in managing their blood pressure levels. A multi-herbal system can address these short comings by simultaneously addressing various blood pressure regulation pathways and therefore the probability of a multi-herbal system to address the blood pressure management deficiencies increases significantly.

### Consuming Many Herbs

While consuming multiple herbs would greatly help with addressing blood pressure management needs of many individuals, the challenges of developing and consuming multiple herbs daily poses several difficulties: 1. Identification of effective herbal formulation of multiple herbs; 2. Most of the herbs could only be effective when consumed in huge quantities lifelong which is humanly not possible; 3. The organoleptics of most natural materials are not suitable for daily consumption to deliver benefits continuously, 4. Achieving patient compliance remains a challenge with herbal formulations. The above are the most primary roadblocks towards development and administration of a multi-herbal formulations from the all the other issues known to a person skilled in the art.

### Quantity of Herbs

The quantity of each of the herbs to deliver the required functionality is often large. For example, separate clinical studies have shown the need to consume 30 g of Flax Seeds to deliver an average of 10 mm Hg reduction in Systolic blood pressure and 7 mm Hg reduction is diastolic blood pressure. Clinical studies and meta data analysis of ginger consumption indicated ginger doses of 3 g/day resulted in systolic blood pressure reduction of 6.36 mm Hg and diastolic blood pressure reduction of 2.12 mm Hg.

Consuming large quantity of herbs and also herbs that have pungent taste like ginger makes it very difficult to comply. This is due to low solubility of active ingredients in water. Most beneficial properties are delivered to a particular organ in a water-soluble form to deliver the desired functionality.

The multi-herbal system developed by the inventors of the present invention, with the active ingredients in a water soluble form, the current invention delivers all the benefits of multiple herbs in a water soluble form that have shown to delivery benefits to a much larger cross section of subjects who may individually require assistance from specific target mechanisms which can differ from individual to individual.

### Herbs & Spices Used in Current Invention

The object of the present invention is to provide a herbal formulation comprising herbs from different families such as Nigella sativa, Cinnamomum verum, Curcuma longa, Zingiber officinale, Elettaria cardamom, Myristica fragrans, Syzygium aromaticum, Psidium guajava, Linum usitatissimum, Cuminum cyminum, Glycyrrhiza glabra, Trigonella foenum-graecum, Foeniculum vulgare.

The antihypertensive effect of Nigella sativa appears to be mediated by a reduction in cardiac oxidative stress and angiotensin-converting enzyme activity, an increase in cardiac heme oxygenase-1 activity and a prevention of plasma nitric oxide loss.

Consumption of Cinnamomum verum (cinnamon) in short term studies was associated with a notable reduction in both Systolic and Diastolic Blood Pressure.

Curcuma Longa (Turmeric) is popular for its antioxidant curcumin which helps in lowering blood pressure. Its antioxidant properties are the responsible for regulating blood pressure in the body. Curcumin also protects against vascular dysfunction in the body. Apart from regulating blood pressure, turmeric also helps in taking care of the damage caused by high blood pressure. A common reason behind heart ailments is accumulation of plaque in arteries. Plaque narrows arteries and slows blood flow to heart, brain and various other body parts. By improving lipids control Curcuma Longa also helps with lowering arterial plaque.

Zingiber officinale (Ginger) is an ancient herb used for treatment of variety of diseases. It is believed to have diuretic properties which lowers blood pressure.

Elettaria cardamomum (Cardamom) seeds and fruit shell contains flavonoids. Clinical studies have shown long term administration of cardamom has significant blood pressure lowering effect along with fibrinolysis and antioxidant enhancing properties in patients with stage 1 hypertension.

Myristica fragrans (Nutmeg) has hypotensive abilities and can help manage hypertension. Also, nutmeg is rich in calcium, potassium and magnesium which are the key nutrients to manage high blood pressure. In addition, the stress relieving properties is believed to help in relaxing the blood vessels which in turn helps in the smooth functioning of the heart and lowering blood pressure.

Syzygium aromaticum (Clove) is a rich source of anti-oxidants which possess anti-inflammatory profiles. These compounds in clove are believed to lower inflammation and arterial stiffness. Further, Clove oil is known to act as a natural blood thinner which also provides blood pressure lowering properties.

Psidium guajava (Guava Leaves) are a rich source of anti-oxidants which can help lower inflammation in the body. Inflammation reduction is associated with reduction in arterial stiffness and corresponding reduction in blood pressure values.

Linum usitatissimum (Flaxseeds) Flaxseed is a rich dietary source of a-linolenic acid, lignans, and fiber, with a number of positive health benefits on blood pressure. Clinical studies have shown consuming 30 g of flaxseeds lowered systolic and diastolic blood pressure by 10 mm and 7 mm HG in six months. Levels of circulating a-linolenic acid levels were correlated with decreases in both systolic and diastolic blood pressure.

Cuminum cyminum (Cumin) is a rich source of potassium and helps to regulate blood pressure by balancing out the negative effects of excess sodium levels.

Glycyrrhiza glabra (Licorice) is a rich source of potent antioxidant, anti-inflammatory, and antimicrobial effects. In traditional Indian medicine systems of Ayurveda and Sidha small amounts of Licorice root is added to enhance the power of synergy between the different herbs in a blend and is used in many classic Ayurvedic formulas as a harmonizing herb. In allopathic systems of medicine, the safe level of Licorice consumption is generally thought to be 2.5 g per day.

Trigonella foenum-graecum (Fenugreek) Fenugreek seeds and leaves are loaded with a high amount of soluble fiber, which aids in decreasing cholesterol level. A diet that is high in fiber has been linked to steady levels of blood pressure.

Foeniculum vulgare (Fennel Seeds) are a rich source of potassium which is beneficial for blood pressure regulation because it counteracts the effects of sodium and regulates the amount of fluid present in the bloodstream. Fennel seeds are also a source of dietary fiber, which reduce inflammation and blood sugar levels, and aid in weight loss, all of which may occur as co-morbid conditions in those with high blood pressure.

### Storage of Herbs & Spices, Grinding and Processes for Enhancing Organoleptic Properties of Herbs and Spices

The storage of herbs and spices under controlled conditions of temperature & humidity, grinding of herbs and organoleptic processes enhancement via making slurry, filtration and distillation, ion exchange and carbon bed adsorption processes are discussed in detail in the Indian Patent application no: 202041041780. The current invention uses the same steps discussed in this application. The formulation and the composition of the multiherb extract for current application in hypertension management is accordingly modified. The processing conditions and ranges specified in the aforementioned patent application remain the same.

### EXAMPLES

The present invention is represented below by the help of representative examples. The examples do not limit the scope of the present invention.

### Mixed Herbs Formulation

Herbs and spices were purchased and stored as per storage conditions specified in the Indian Patent application no: 202041041780. The following formulation 1 summarized in Table 2 and covers the formulation used in the human clinical trial to demonstrate the benefits in terms of lowering systolic and diastolic hypertension among hypertensive subjects.

TABLE 2
Representative Formulation

| Scientific Name | Formulation |
|---|---|
| Nigella sativa | 1.50% |
| Cinnamomum verum | 2.50% |
| Curcuma longa | 1.0% |
| Zingiber officinale | 3.00% |
| Elettaria cardamom | 3.20% |
| Myristica fragrans | 2.80% |
| Syzygium aromaticum | 3.00% |
| Psidium guajava | 43.50% |
| Linum usitatissimum | 31.50% |
| Cuminum cyminum | 4.50% |
| Glycyrrhiza glabra | 1.50% |
| Trigonella foenum-graecum | 1.00% |
| Foeniculum vulgare | 1.00% |

### Example 1

Mixed herbal powder Formulation 1 summarized in Table 2 was used in the test. 14 kg of this mixed herbal powder was dispersed in 56 litres of water. The slurry was kept in suspension with a motorized impeller for 12 hours. The resultant slurry was charged to a 100 litre glass batch distillation still along condenser with a 2.5 sq m condensing surface area. The condenser was water cooled where water circulating at 3 deg C inlet and 5 deg c outlet temperature. The glass vessel was immersed in a 50/50 ethylene glycol batch and heat input to the ethylene glycol was provided by an electrical heating element which was set at 150 deg C. The process was operated for 5 hours at a vacuum of 0.1 mm Hg (0.1 torr) and 30 litres of the herbal concentrate was collected. The boiling point of the batch was 41 deg C. Overall process yield from herbal water to herbal condensate was 53.6%. The herbal condensate was crystal clear and free of any color.

Herbal extract produced was used as an adjunctive supplement in hypertension management as the ingredients used in the formulation are well known to be effective in hypertensives. A single centric, open-label study to evaluate the efficacy of Hypertension Herbal Solution in hypertensive patients was undertaken. A total of 50 (25-subjects new hypertensive i.e <1 Year with an average time of diagnosis of HTN of 8 months; 25 subjects with long term Hypertensive i.e >2 years with an average time from diagnosis of HTN of 7.75 years) subjects with age group >45 years and surviving with standard medication should be considered to initiate treatment with Herbal extract.

The study was conducted in accordance with the principles of “The Declaration of Helsinki” (World Medical Association) and Good Clinical Practice (GCP) Guidelines issued by the ICMR & DCG(I), Govt. of India. All participants were explained about the study in details and were provided opportunity to raise any queries and doubts about the study. Informed written consent was obtained from all patients/subjects before starting any study related procedures on the patients.

### Inclusion Criteria:

Subjects meeting with following criteria to be included in the study:
1. Participants willing to sign an Informed Consent Form indicating that they understand the purpose of and the procedures required for the trial and are willing to participate in it
2. Male and female treatment subjects, aged >45 years, with diagnosis of hypertension (DBP 110 mm Hg and SBP >150mm Hg) at entry, who are considered appropriate for the herbal extract supplement

### Exclusion Criteria:

Subjects meeting with following criteria to be excluded from the study:
1. Patients not ready to refrain from taking treatment with other drugs that could interfere with the evaluation of efficacy and tolerability.
2. Patients with secondary hypertension (eg. due to renal artery stenosis etc.)
3. Patients with uncontrolled diabetes mellitus, significant cardiovascular or cerebrovascular disease [congestive heart failure (New York Heart Association class III/IV); unstable angina in the previous 3 months; stroke within the previous 6 months; myocardial infarction, cardiac surgery, or percutaneous transluminal coronary angiography in the previous 3 months; cardiac arrhythmia; cardiomyopathy; aortic or mitral valve stenosis]
4. Patients with renal or hepatic disease (based on known history or lab abnormalities) or serum electrolyte abnormalities
5. Pregnant or breast-feeding women, and women of childbearing age not using an adequate method of contraception
6. Presence of any other clinically significant disease or laboratory findings that in the Investigator's opinion may affect efficacy or safety assessments.
7. History of alcohol or drug abuse, or suspicion thereof in the Investigator's judgment.
8. History of or active hepatitis B or C within the past 3 months or history of HIV infection.
9. History of seizure disorder or epilepsy
10. Participants with acute or severe bronchial asthma or hypercapnia.
11. Participation in another trial concurrently or within 4 weeks prior to the Screening Visit.
12. Known to or suspected of not being able to comply with the protocol and the use of the investigational medicinal product.

The duration of this study was 6 months (180-days). The subjects continued their standard medications and supplemented the medication with the herbal extract. Each subject consumed 7.5 ml of the herbal extract mixed with 500 ml of water. This herbal extract mixed with water was consumed in equal proportions during breakfast, lunch and dinner. The following summarizes the visits made by each patient during the course of the clinical trial

Visit 0: Screening (day -2 to day 0)

Visit 1: Enrolment & Treatment Allocation (day 0)

Visit 2: Follow-up (Week 4)

Visit 3: Study completion (Week 12)

Visit 4: Study completion (Week 24)

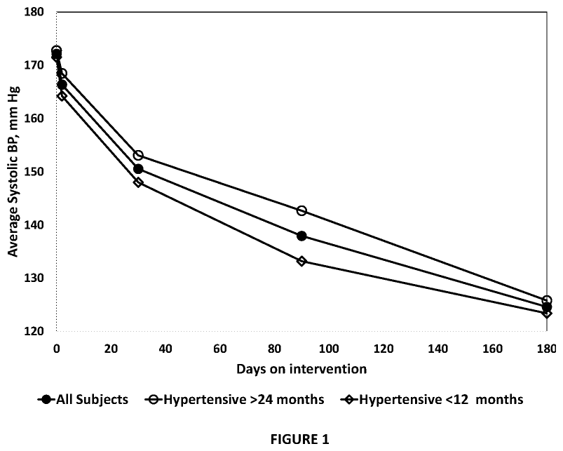

Results are demonstrated in the following FIGS. 1-6 and tables 3-7 highlighting synergism and the significant therapeutic effect achieved obtained by the present formulation:

FIG. 1 is plot of the average systolic blood pressure of all subjects, old and newly diagnosed hypertensives during the course of the clinical trial.

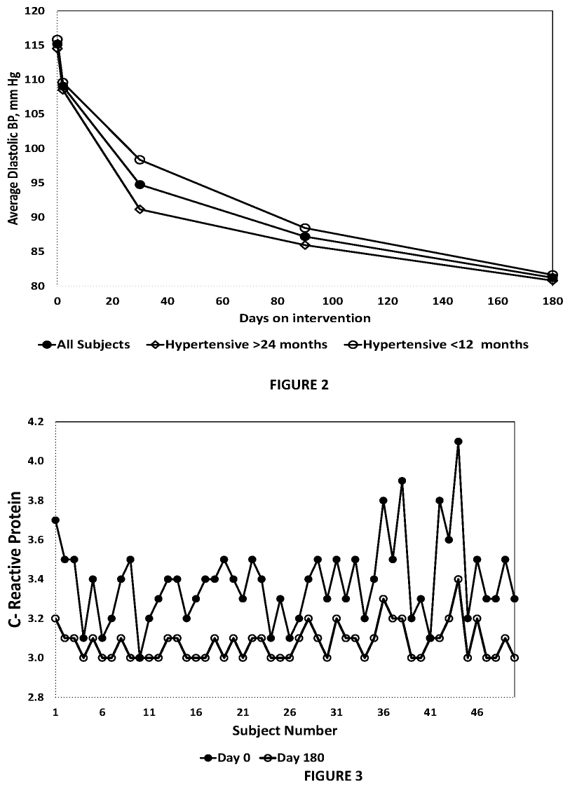

FIG. 2 is plot of the average systolic blood pressure of all subjects, old and newly diagnosed hypertensives during the course of the clinical trial.

FIG. 3 is plot of the C-Reactive protein of all subjects at the start of the clinical trial and at the end of the clinical trial (day 180). It shows consistent lowering of C-reactive protein levels which indicate lowering of inflammation levels which have a positive benefit with respect to lowering of arterial stiffness and a corresponding lowering of systolic and diastolic blood pressure values.

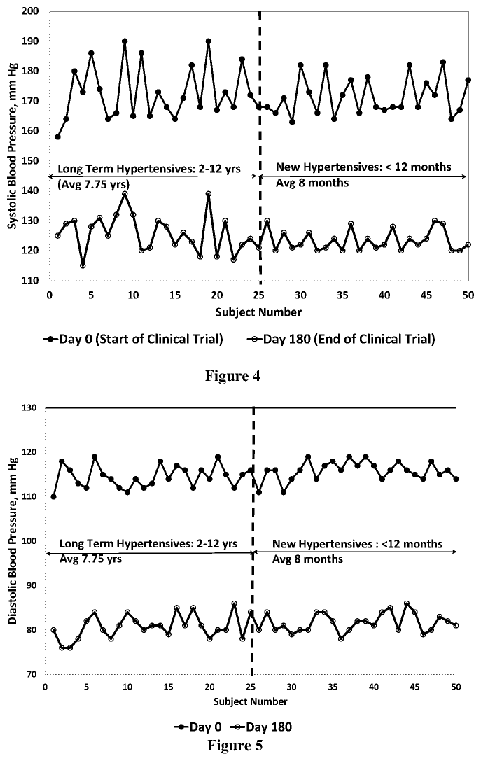

FIG. 4 is plot of systolic blood pressure of all subjects at the start of the clinical trial (Day 0) and at the end of the clinical trial (day 180). The data clearly demonstrates the reduction in systolic blood pressure for all subjects. Further, all subjects at the day 180 are moving close to the ideal diastolic blood pressure values of 120 mm Hg. Average systolic blood pressure of all subjects was 172 mm Hg at day 0 and averaged 123 mm Hg at the end of the clinical trial at day 180.

FIG. 5 is plot of diastolic blood pressure of all subjects at the start of the clinical trial (Day 0) and at the end of the clinical trial (day 180). The data clearly demonstrates the reduction in diastolic blood pressure for all subjects. Further, all subjects at the day 180 are moving close to the ideal diastolic blood pressure values of 80 mm Hg. Average systolic blood pressure of all subjects was 115 mm Hg at day 0 and averaged 82 mm Hg at the end of the clinical trial at day 180.

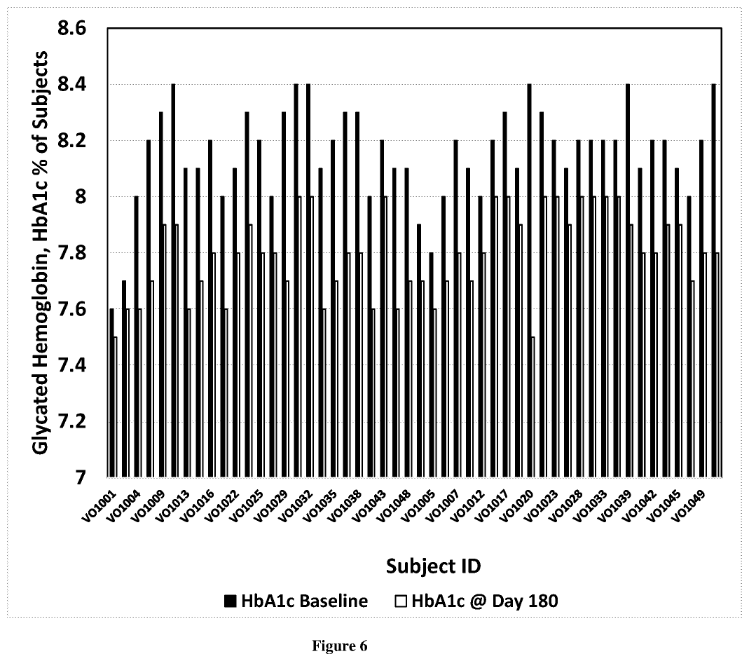

FIG. 6 is plot of the glycated hemoglobin (HbA1c) value for all subjects at the start of the clinical trial, at day 90 and and ay day 180, the end of the clinical trial. The data shows every subject is seen to have reduction in HbA1c thereby indicating the formulation to possesses both BP lowering properties as well as long term blood glucose lowering properties.

TABLE 3
Average Systolic and Diastolic Blood pressure values and
reductions of subjects from 6-month clinical trial

| | Average Systolic BP, mm Hg | | | | | Average Diastolic BP, mm Hg | | | | |
|---|---|---|---|---|---|---|---|---|---|---|
| Days on Intervention | 0 | 2 | 30 | 90 | 180 | 0 | 2 | 30 | 90 | 180 |
| All Subjects | 172 | 166 | 151 | 138 | 125 | 115 | 109 | 95 | 87 | 81 |
| Hypertensive > 24 months | 173 | 168 | 153 | 143 | 126 | 115 | 109 | 91 | 86 | 81 |
| Hypertensive < 12 months | 172 | 164 | 148 | 133 | 123 | 116 | 110 | 98 | 88 | 82 |

| | Average Delta in Systolic, mm Hg | | | | | Average Delta in Diastolic, mm Hg | | | | |
|---|---|---|---|---|---|---|---|---|---|---|
| Days on Intervention | 0 | 2 | 30 | 90 | 180 | 0 | 2 | 30 | 90 | 180 |
| All Subjects | | −6 | −22 | −34 | −48 | | −6 | −20 | −28 | −34 |
| Hypertensive > 24 months | | −4 | −20 | −30 | −47 | | −6 | −23 | −29 | −34 |
| Hypertensive < 12 months | | −7 | −24 | −38 | −48 | | −6 | −17 | −27 | −34 |

Table 3 summarizes the systolic and diastolic blood pressure values for all subjects. The data has also been analyzed based on new versus old hypertensives and shows the intervention works equally well between new and old hypertensives. The average systolic and diastolic blood pressure reduction of all subjects at the end of the clinical trial was 48 mm Hg and 37 mm Hg respectively.

TABLE 4
Average Systolic and Diastolic Blood pressure values and reductions of subjects
from 6-month clinical trial as a function of medications consumed by subjects

| Medication Type | No. of | Average Systolic BP, mm Hg | | | | | Average Diastolic BP, mm Hg | | | | |
|---|---|---|---|---|---|---|---|---|---|---|---|
| Days on Intervention | Subjects | 0 | 2 | 30 | 90 | 180 | 0 | 2 | 30 | 90 | 180 |
| ARB | 1 | 166 | 160 | 152 | 140 | 132 | 114 | 105 | 92 | 86 | 78 |
| ARB + CCB | 3 | 174 | 165 | 148 | 134 | 126 | 117 | 111 | 95 | 90 | 84 |
| BB | 6 | 170 | 168 | 155 | 139 | 124 | 116 | 109 | 94 | 88 | 81 |
| CCB | 8 | 171 | 165 | 149 | 136 | 124 | 115 | 111 | 98 | 88 | 81 |
| CCB + BB | 4 | 172 | 169 | 152 | 137 | 124 | 114 | 109 | 95 | 89 | 84 |
| DU + ARB | 28 | 173 | 166 | 150 | 139 | 125 | 115 | 108 | 94 | 86 | 81 |

| | | Average Delta Systolic, mm Hg | | | | | Average Delta Diastolic, mm Hg | | | | |
|---|---|---|---|---|---|---|---|---|---|---|---|
| Days on Intervention | | 0 | 2 | 30 | 90 | 180 | 0 | 2 | 30 | 90 | 180 |
| ARB | 1 | | −6 | −14 | −26 | −34 | | −9 | −22 | −28 | −36 |
| ARB + CCB | 3 | | −9 | −26 | −40 | −48 | | −6 | −22 | −27 | −33 |
| BB | 6 | | −2 | −16 | −32 | −46 | | −7 | −22 | −28 | −35 |
| CCB | 8 | | −6 | −22 | −34 | −47 | | −4 | −17 | −27 | −34 |
| CCB + BB | 4 | | −3 | −20 | −35 | −49 | | −5 | −19 | −25 | −30 |
| DU + ARB | 28 | | −7 | −23 | −34 | −48 | | −7 | −21 | −29 | −34 |

NOTE:
Medication Abbreviations
ARB = Angiotensin Receptor Blocker
CCB = Calcium Channel Blocker
BB = Beta Blocker
DU = Diuretics

Table 4 summarises the systolic and diastolic blood pressure values for all subjects expressed as a function of the various medications they continued to take during the course of this clinical trial. The average systolic and diastolic blood pressure reduction was seen to work equally well regardless of the medications being consumed by the subjects.

TABLE 5
Average Systolic and Diastolic Blood Pressure Reductions in mm Hg of herbal extract used
in current invention versus other herbal powders and extracts published in literature

| | | | | Duration | | |
|---|---|---|---|---|---|---|
| | | Average Systolic | Average Diastolic | of Inter- | | |
| Herb | Quantity Consumed | Reduction, mm Hg | Reduction, mm Hg | vention | | Reference |
| Herbal | 7.5 ml of herbal water | Day 2: 6.0 mm hg | Day 2: 6.0 mm hg | 6 | months | Example 1 |
| Extract | produced from 2.5 g of | Day 30: 22.0 mm Hg | Day 30: 20.0 mm Hg | | | |
| Formu- | herbal powder | Day 90: 34.0 Hg | Day 90: 28.0 Hg | | | |
| lation | | Day 180: 48.0 mm Hg | Day 180: 34.0 mm Hg | | | |
| Flax Seeds | 30 g/day | Day 30: 6.0 mm Hg | 7.00 | 6 | months | Rodriguez-Leyva D, Weighell W, Edel AL, |
| | | Day 60: 6.3 mm Hg | | | | LaVallee R, Dibrov E, Pinneker R, Maddaford |
| | | Day 180: 6.7 mm Hg | | | | TG, Ramjiawan B, Aliani M, Guzman R, Pierce |
| | | | | | | GN. Potent antihypertensive action of dietary |
| | | | | | | flaxseed in hypertensive patients. |
| | | | | | | Hypertension. 2013 December; 62(6): 1081-9. |
| | | | | | | doi: 10.1161/HYPERTENSIONAHA.113.02094. |
| | | | | | | Epub 2013 Oct. 14. PMID: 24126178. |
| Ginger | ≥3.0 g/day | 6.36 | 2.12 | ≤8 | weeks | Hasani H, Arab A, Hadi A, Pourmasoumi M, |

| | | | | | |
|---|---|---|---|---|---|
| | | | | | Ghavami A, Miraghajani M. Does ginger |
| | | | | | supplementation lower blood pressure? A |
| | | | | | systematic review and meta-analysis of |
| | | | | | clinical trials. Phytother Res. 2019 June; 33(6) |
| | | | | | 1639-1647. doi: 10.1002/ptr. 6362. PMID: |
| | | | | | 30972845. |
| Cinnamon | Day 30: 85 mg Extract | Day 30: 6.0 mm Hg | Day 30: 4.1 mm Hg | 3 month | Ranasinghe P, Jayawardena R, Pigera S, et al. |
| Extract | Day 60: 250 mg Extract | Day 60: 6.3 mm Hg | Day 60: 4.9 mm Hg | study | Evaluation of pharmacodynamic properties |
| (Healthy | Day 90: 500 mg Extract | Day 90: 6.7 mm Hg | Day 90: 4.8 mm Hg | | and safety of Cinnamomum zeylanicum |
| Adults) | 85 mg produced from 1 g | | | | (Ceylon cinnamon) in healthy adults: a phase I |
| | Cinnamon | | | | clinical trial. BMC Complement Altern Med. |
| | 250 mg produced from | | | | 2017; 17(1): 550. Published 2017 Dec. 28. |
| | 3 g Cinnamon | | | | doi: 10.1186/s12906-017-2067-7 |
| | 500 mg produced from | | | | |
| | 6 g Cinnamon | | | | |

Table 5 compares the clinical trial data summarized in Example 1 versus clinical trial data from other herbal treatments. The herbal extract described in example 1 is found to cause a significantly higher lowering in both systolic and diastolic blood pressure values than single component herbs. The formulation of example 1 has demonstrated highly significant therapeutic effect obtained by the synergistic effect of the formulation.

TABLE 6
Average glycated hemoglobin (HbA1c) of the clinical trial participants
at the star (day 0) and at the end (day 180) of the clinical trial

| | Day 0 | Day 90 | Day 180 |
|---|---|---|---|
| HbA1c, Average | 8.2% | 8.0% | 7.8% |
| % HbA1c Reduction | | −1.86% | −4.34% |

Table 6 summarises the glycated hemoglobin (HbA1c) value for all subjects at the start of the clinical trial, at day 90 and and ay day 180, the end of the clinical trial. HbA1c values averaged 8.2% at the start of the trial and reduced to 8.0% at day 90 and 7.8% at day 180 of the clinical trial. Further, the HbA1c reduction was noted among all of the 50 subjects in the clinical trial.

### Example 2

### Study Demonstrating the Effect of the Herbal Formulation on Heart Endurance

A 57-year old male who suffered a heart attack three years ago has been put of a regimen of drug cocktails to control the heart rate and vital parameters including pulse rate, heart pumping efficiency. Among various medication, the subject was prescribed Metaprorol, a beta blocker class of drug to control heart rate. However, the subject was experiencing a significant reduction in pulse rate much below 50 and his Metaprorol was discontinued. The immediate side effect of withdrawing from this drug was increase in blood pressure which increased to 155/110 mg. The subject was put on Lozarton 25 mg, an Angiotensin II receptor blocker class of drug to lower blood pressure. The subject was advised to increase the dosage of Losartan from 25 mg to 40 mg.

In consultation with the physician, the subject supplemented Losartan 25 mg with the herbal water described in Example 1. 15 ml of herbal water mixed in 500 ml of water daily for a period 6 months and consumed throughout the day. The subject in question has noted that within 15 minutes of consuming the first 50 ml of the herbal water, his BP values are lowered from the 155/110 mm to 120/80 mm and remained at this level throughout the day.

The subject also has found a marked improvement in endurance. The subject follows a strict exercise regimen and follows his pulse rate carefully during daily morning walks and maintains his walking pace in order to ensure pulse rate stays below 130 beats per minute (bpm). Prior to use of herbal waters, the subjects would clock 3.7 miles per hour while keeping the pulse rate below 130 bpm. After consuming the herbal water for six months, the subject is able to increase the pace of his walks to 4.5 miles per hour while keeping the heart rate well below the target 130 bpm. This translates to an increase in walking speed of 21.6% while keeping pulse rate well below physician recommended value of less than 130 bpm. This clearly demonstrated building of endurance from lower BP most likely from reduction in atherosclerosis which caused stiffening of arteries. In six months' time endurance increased by 21.6% while keeping pulse rate well below the target levels of 130 bpm.

By combining multiple herbs into an easily consumable water format with good organoleptic properties in addition to good functional properties, it allows easy consumption of the herbal waters on a daily basis by hypertensive subjects.

The significant lowering of systolic and diastolic blood pressure levels has very positive outcomes with respect to long term stroke reduction among hypertensive subjects. High blood pressure is the most important global risk factor for death and disability. Systematic review and meta-analysis by many global health organizations confirm that lowering blood pressure in people with hypertension significantly reduces the risk of cardiovascular events and deaths, and leads to improved health outcomes. According to Ettehad D, Emdin C A, Kiran A, et al. Blood pressure lowering for prevention of cardiovascular disease and death: a systematic review and meta-analysis. Lancet. 2015, Every 10 mm Hg systolic blood pressure reduction reduced the risk of major cardiovascular events by 20%, coronary heart disease by 17%, stroke by 27%, heart failure by 28%, and death from all causes by 13%. With average reduction of systolic & diastolic blood pressure of 48 mm Hg and 34 mm Hg respectively, the formulation and water soluble characteristics of the herbal extract clearly shows the substantial benefits that are accrued with the current invention.

Among the clinical trial participants, all subjects selected were also diabetic. The average glycated hemoglobin or HbA1c of the clinical trial participants was 8.2% and reduced to 7.8% by the end of the clinical trial as summarized in Table 6. The herbal solution administered also lowered HbA1c levels in each of the clinical trial participants as shown in FIG. 6.

The coexistence of diabetes and hypertension is known to have a multiplicative effect on adverse clinical outcomes with respect to both microvascular and macrovascular disease. High blood pressure is twice more likely to strike a person with diabetes than a person without diabetes. Left untreated, high blood pressure can lead to heart disease and stroke. In fact, a person with diabetes and high blood pressure is four times more likely to develop heart disease than someone who does not have either of the conditions. Effective management of diabetes should therefore include a multifaceted approach combining optimal control of blood pressure and lipids with appropriate glycemic control. By simultaneously addressing long term blood sugar control as evidenced by HbA1c reduction among the hypertensive in this study, the current invention appears to serve as a combination or dual therapy for hypertension & diabetes thereby addressing the multiplicative risks.
1. In the present study, the positive effect of the formulation was analyzed from several other aspects: On Atherosceloris & CRP

C-reactive protein (CRP) is a protein the liver produces in the presence of infection or inflammatory disease. When body has an infection, the white blood cells act to fight it by producing a number of proteins, some of which stimulate the liver to produce CRP. The blood level of CRP has been used for many years to evaluate the level of inflammation or infection.

The level of C-reactive protein can be an indicator of risk for developing cardiovascular problems. This is because the development of atherosclerosis (laying down of cholesterol inside the blood vessel walls) is associated with inflammation within the vessel walls. The result is higher levels of CRP in patients with atherosclerosis than in those without atherosclerosis.

Data summarized in FIG. 3 clearly shows reduction in CRP among majority of subjects and thereby suggesting lowering of arterial inflammation.

### 2. Blood Viscosity

Blood viscosity (BV) is an important blood property, and plays a key role in maintaining vascular homeostasis. Homeostasis is the state of steady internal, physical, and chemical conditions maintained by living systems. This is the condition of optimal functioning for the organism and includes many variables, such as body temperature and fluid balance, being kept within certain pre-set limits.

Studies of blood viscosity among normal and hypertensive patients indicated a strong correlation between blood viscosity and blood pressure as summarized in the following study: Letcher R L, Chien S, Pickering T G, Sealey J E, Laragh J H. Direct relationship between blood pressure and blood viscosity in normal and hypertensive subjects. Role of fibrinogen and concentration. Am J Med. 1981 June;70(6):1195-1202. doi: 10.1016/0002-9343(81)90827-5. PMID: 7234890.

As summarized in table 3, the systolic and diastolic values of clinical trial participants lowered on an average by 6 mm each by day 2 of the clinical trial, which is a highly significant value. Such significant changes in blood pressure are likely coming from blood viscosity lowering mechanisms rather than physiological mechanisms which tend to take a much longer time to set processes in motion.

### 3. Endurance Enhancements

As BP reduction is achieved, it is likely that arterial stiffness is reduced and the heart can pump blood more efficiently. These improvements in efficiencies have a direct bearing on an individuals' endurance parameters in terms of maintaining or increasing the exercise regimen without increasing the heart rate.

### 4. Cardiac Oxidative Stress & Angiotensin-Converting Enzyme Activity-,:

The antihypertensive effect of the current herbal formulation appears to be mediated by a reduction in cardiac oxidative stress and angiotensin-converting enzyme activity, an increase in cardiac heme oxygenase-1 activity and a prevention of plasma nitric oxide loss.

### 5. Efficacies of Current Pharma Interventions and Use of Combination Therapies

When hypertensive patients do not achieve adequate control of their blood pressure, the options to try and achieve required treatment goals are to increase the dose of monotherapy (which increases the risk of side effects) or to use drug combinations with minimum side effects. In order to avoid complications, it is important to start treatment as soon as possible, achieve the goals in the shortest time possible and ensure treatment adherence.

The mechanisms that lead to a blood pressure increase in a patient are diverse-monotherapy acts on one or at best two of these mechanisms, while the use of combinations of drugs allows for action on several different hypertensive mechanisms. By combining two drugs with different mechanisms of action, an antihypertensive effect that is significantly greater than that obtained by monotherapy is possible. This the one of the primary reasons that organizations such as American Heart Association recommends combination therapies to improve BP reduction and lower cardio vascular risk factors.

TABLE 7

| | Avg Daily | Systolic | Diastolic |
|---|---|---|---|
| | Dosage, mg or | Reduction, mm | Reduction, |
| Class of Drug | herbal water in ml | Hg | mm Hg |
| Thiazide | | −11.2 | −9.08 |
| Beta Blockers | | −12.9 | −10.3 |
| CCB-DHP | | −10.4 | −8.7 |
| CCB-No DHP | | −9.2 | −8.8 |
| ACE | | −11.4 | −8.7 |
| ARB | | −12.9 | −8.5 |
| ARB/CCB | 35/9.2 | −23.7 | −7.4 |
| ARB/DU | 26.2/17.8 | −22.2 | −14.7 |
| CCB/BB | 10/100 | −20.8 | −13.3 |
| ARB/DU | 189.1/16.3 | −20.2 | −11.8 |
| ACE/DU | 3.6/1.0 | −15.0 | −10.7 |
| ARB/DU | 100/25 | −14.4 | −13.2 |
| ARB/CCB | 156.5/4.2 | −13.0 | −5.4 |
| All Combinations Avg | | −20.2 | −12.8 |
| Herbal Waters - | 15 ml/day | −47.5 | −33.9 |
| Current Specification | | | |

Table 7 compares reported reduction ranges for various single molecule pharma drug therapies, combination therapies relative to the multi-component herbal formulation from the current system. The data presented here clearly demonstrates the benefits of the current herbal formulation which is delivering significant efficacy with food grade herbs and spices which do not come with side effects which are very prevalent with pharma therapies.

Reference: Ref: Paz M A, de-La-Sierra A, Saez M, et al. Treatment efficacy of anti-hypertensive drugs in monotherapy or combination: ATOM systematic review and meta-analysis of randomized clinical trials according to PRISMA statement. Medicine (Baltimore). 2016;95(30):e4071. doi:10.1097/MD.0000000000004071

In the multi-component herbal formulation disclosed in the current invention, various herbs and spices have certain predominant mechanism of action. By combining multiple herbs and spices, multiple mechanisms of action are addressed simultaneously. Just as in combination therapies used in pharmaceutical/allopathic interventions, use of a multi-component system with food based herbs and spices we are able to greatly increase efficacy (the reduction in the specific response, i.e., systolic and diastolic blood pressure values) and effectiveness (the percent of the population seeing benefits). As we can see in the examples summarized in the present invention, the multi-component herbal formulation resulted in 100% of the clinical study population seeing a positive response with respect to lowering their BP values.

Although the invention has been described with reference to specific embodiments, this description is not meant to be construed in a limiting sense as it will become apparent to persons skilled in the art upon reference to the description of the invention. It is therefore, contemplated that such modifications can be made without departing from the spirit or scope of the present invention as defined.

1. A herbal formulation comprising, a therapeutically effective amount extracs from plant material selected from plants from the group consisting of Nigella sativa, Cinnamomum verum, Curcuma longa, Zingiber officinale, Elettaria cardamom, Myristica fragrans, Syzygium aromaticum, Psidium guajava, Linum usitatisiumum, Cuminum cyminum, Glycyrrhiza glabra, Trigonella foenum-graecum, Foeniculum vulgar and combinations thereof.

2. A herbal formulation as claimed in claim 1, comprising:

| | |
|---|---|
| Nigella sativa | 0-10.0% |
| Cinnamomum verum | 0-5.0%, |
| Curcuma longa | 0-7.0%, |
| Zingiber officinale | 1.0-7.75%, |
| Elettaria cardamom | 0.5-10%, |
| Myristica fragrans | 2.0-10.5%, |
| Syzygium aromaticum | 1.25-9.5%, |
| Psidium guajava | 10.0-65.0%, |
| Linum usitatissimum | 8.0-55.0%, |
| Cuminum cyminum | 0.5-12.5%, |
| Glycyrrhiza glabra | 0.5-1.5%, |
| Trigonella foenum-graecum | 0-6.5%, and |
| Foeniculum vulgare | 0-7.5% |

by weight of the herbal formulation.

3. The herbal formulation as claimed in claim 2, wherein the plant material comprises Nigella sativa seeds, Cinnamomum verum seeds, Curcuma longa root, Zingiber officinale root, Elettaria cardamom seeds, Myristica fragrans seeds, Syzygium aromaticum flower buds, Psidium guajava leaves, Linum usitatissimum seeds, Cuminum cyminum seeds, Glycyrrhiza glabra root, Trigonella foenum-graecum seeds, Foeniculum vulgare seeds.

4. The herbal formulation as claimed in claim 2, comprising:

| | |
|---|---|
| Nigella sativa | 1.5%, |
| Cinnamomum verum | 2.5%, |
| Curcuma longa | 1.0%, |
| Zingiber officinale | 3.0%, |
| Elettaria cardamom | 3.2%, |
| Myristica fragrans | 2.8%, |
| Syzygium aromaticum | 3.0%, |
| Psidium guajava | 43.5%, |
| Linum usitatissimum | 31.5%, |
| Cuminum cyminum | 4.5%, |
| Glycyrrhiza glabra | 1.5%, |
| Trigonella foenum-graecum | 1.2%, and |
| Foeniculum vulgare | 1.3% |

by weight of the herbal formulation.

5. A method of treatment comprising:
administering to a subject in need thereof an effective amount of the herbal formulation as claimed in claim 1,
wherein the herbal formulation treats and/or manages one or more of hypertension, atherosclerosis, arterial stiffness, HbA1c levels, blood level of CRP, vascular homeostasis, cardiac oxidative stress, blood viscosity, heart endurance. angiotensin-converting enzyme activity, and combinations thereof.

6. (canceled)

7. The method as claimed in claim 5, further comprising administering a diabetic and/or hypertension drugs.

8. The method as claimed in claim 5, wherein the herbal formulations treats hypertensive and diabetes.

9. The method as claimed in claim 6, wherein the herbal formulation is configured to lower HbA1c levels.

10. The method as claimed in claim 5, wherein the herbal formulation is configured to lower C-reactive protein levels in blood.

11. The method as claimed in claim 5, wherein the herbal formulation is configured to improve and maintain blood viscosity.

12. The method as claimed in claim 5, wherein systolic and diastolic values of clinical trial participants lowered on an average by 6 mm each by day 2 of the clinical trial.

13. The method as claimed in claim 5, wherein the herbal formulation is configured improve and maintain heart endurance.
